(19) United States
(12) Patent Application Publication
(10) Pub. No.: US 2009/0088439 A1
(43) Pub. Date: Apr. 2, 2009

(54) Diazinane Compounds

(76) Inventors: Leifeng Cheng, Molndal (SE); Maria Jonforsen, Molndal (SE); Peter Schell, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 12/238,937

(22) Filed: Sep. 26, 2008

Related U.S. Application Data
(60) Provisional application No. 60/975,532, filed on Sep. 27, 2007.

(51) Int. Cl. A61K 31/4985; C07D 475/02; A61P 1/04

(52) U.S. Cl. 514/249; 544/257

(57) ABSTRACT

The present invention relates to novel compounds of the general formula (I)

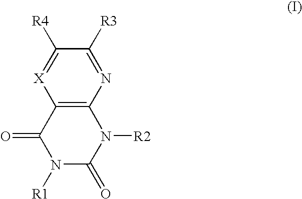

wherein R¹, R², R³ and R⁴ are as defined, having a positive allosteric GABA_{B} receptor (GBR) modulator effect, methods for the preparation of said compounds and to their use, optionally in combination with a GABA_{B} agonist, for the inhibition of transient lower esophageal sphincter relaxations, for the treatment of gastroesophageal reflux disease, as well as for the treatment of functional gastrointestinal disorders and irritable bowel syndrome (IBS).

### FIELD OF THE INVENTION

The present invention relates to novel compounds having a positive allosteric GABA_{B} receptor (GBR) modulator effect, methods for the preparation of said compounds and their use for the inhibition of transient lower esophageal sphincter relaxations, for the treatment of gastroesophageal reflux disease, as well as for the treatment of functional gastrointestinal disorders and irritable bowel syndrome (IBS).

### BACKGROUND OF THE INVENTION

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastroesophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, recent research (e.g. Holloway & Dent (1990) Gastroenterol. Clin. N. Amer. 19, pp. 517-535) has shown that most reflux episodes occur during transient lower esophageal sphincter relaxations (TLESR), i.e. relaxations not triggered by swallows. It has also been shown that gastric acid secretion usually is normal in patients with GERD.

Consequently, there is a need for a therapy that reduces the incidence of TLESR and thereby prevents reflux.

GABA_{B}-receptor agonists have been shown to inhibit TLESR, which is disclosed in WO 98/11885 A1.

Functional gastrointestinal disorders, such as functional dyspepsia, can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II. Functional Gastrointestinal Disorders: Diagnosis, Pathophysiology and Treatment. 2 ed. McLean, Va. Degnon Associates, Inc.; 2000.351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

Irritable bowel syndrome (IBS) can be defined in accordance with Thompson W G, Longstreth G F, Drossman D A, Heaton K W, Irvine E J, Mueller-Lissner S A. C. Functional Bowel Disorders and Functional Abdominal Pain. In. Drossman D A, Talley N J, Thompson W G, Whitehead W E, Coraziarri E, eds. Rome II: Functional Gastrointestinal Disorders. Diagnosis, Pathophysiology and Treatment. 2 ed. McLean, Va. Degnon Associates, Inc.; 2000.351-432 and Drossman D A, Corazziari E, Talley N J, Thompson W G and Whitehead W E. Rome II: A multinational consensus document on Functional Gastrointestinal Disorders. Gut 45 (Suppl. 2), II1-II81.9-1-1999.

GABA_{B} Receptor Agonists

GABA (4-aminobutanoic acid) is an endogenous neurotransmitter in the central and peripheral nervous systems. Receptors for GABA have traditionally been divided into GABA_{A} and GABA_{B} receptor subtypes. GABA_{B} receptors belong to the superfamily of G-protein coupled receptors (GPCRs).

The most studied GABA_{B} receptor agonist baclofen (4-amino-3-(p-chlorophenyl)butanoic acid; disclosed in CH 449046) is useful as an antispastic agent. EP 356128 A2 describes the use of the GABA_{B} receptor agonist (3-aminopropyl)methylphosphinic acid for use in therapy, in particular in the treatment of central nervous system disorders.

EP 463969 A1 and FR 2722192 A1 disclose 4-aminobutanoic acid derivatives having different heterocyclic substituents at the 3-carbon of the butyl chain. EP 181833 A1 discloses substituted 3-aminopropylphosphinic acids having high affinities towards GABA_{B} receptor sites. EP 399949 A1 discloses derivatives of (3-aminopropyl)methylphosphinic acid, which are described as potent GABA_{B} receptor agonists. Still other (3-aminopropyl)methylphosphinic acids and (3-aminopropyl)phosphinic acids have been disclosed in WO 01/41743 A1 and WO 01/42252 A1, respectively. Structure-activity relationships of several phosphinic acid analogues with respect to their affinities to the GABA_{B} receptor are discussed in J. Med. Chem. (1995), 38, 3297-3312. Sulphinic acid analogues and their GABA_{B} receptor activities are described in Bioorg. & Med. Chem. Lett. (1998), 8, 3059-3064. For a more general review on GABA_{B} ligands, see Curr. Med. Chem.—Central Nervous System Agents (2001), 1, 27-42.

Positive Allosteric Modulation of GABA_{B} Receptors

2,6-Di-tert-butyl-4-(3-hydroxy-2,2-dimethylpropyl)phenol (CGP7930) and 3-(3,5-di-tert-butyl-4-hydroxyphenyl)-2,2-dimethylpropanal (disclosed in U.S. Pat. No. 5,304,685) have been described to exert positive allosteric modulation of native and recombinant GABA_{B} receptor activity (Society for Neuroscience, 30^{th} Annual Meeting, New Orleans, La., Nov. 4-9, 2000: Positive Allosteric Modulation of Native and Recombinant GABA_{B} Receptor Activity, S. Urwyler et al.; Molecular Pharmacol. (2001), 60, 963-971).

N,N-Dicyclopentyl-2-methylsulfanyl-5-nitro-pyrimidine-4,6-diamine has been described to exert positive allosteric modulation of the GABA_{B} receptor (The Journal of Pharmacology and Experimental Therapeutics, 307 (2003), 322-330).

Pteridine Derivatives

Liebigs Annalen der Chemie, 1984, 1798-1814, discloses acylations of lumazines by radical nucleophilic substitution.

EP1380298 (Bristol Myers Squibb Pharma Co) discloses tetrahydropteridines and pyridylpiperazines for treatment of neurological disorders, anorexa, inflammation.

WO2007061360 A2 (AstraZeneca AB) discloses novel bicyclocarbonylaminopyridine-2-carboxamides or 3-bicyclocarbonylaminopyrazine-2-carboxamides.

WO 9620710 (The Regents of the University of California) discloses compounds for inhibition of ceramide-mediated signal transduction. This document discloses in Table 1 a compound (43e) whose structure may be the one of 1,3-dibenzylpteridine-2,4(1H,3H)-dione.

### OUTLINE OF THE INVENTION

The present invention provides a compound of the formula

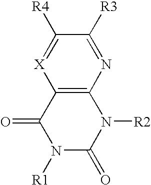

wherein:

R¹ is selected from aryl-C₁-C₁₀-alkyl substituted by halogen;

R² is selected from C₁-C₁₀-alkyl unsubstituted or substituted by one or more of oxo, hydroxy, halogen and tri-C₁-C₁₀-alkylsilyl; C₂-C₁₀-alkenyl; C₃-C₁₀-cycloalkyl-C₁-C₁₀-alkyl; aryl-C₁-C₁₀-alkyl unsubstituted or substituted by one or more of C₁-C₁₀-alkoxy, halogen, cyano and aroyl; and heteroaryl-C₁-C₁₀-alkyl;

R³ is selected from hydrogen and C₁-C₁₀-alkyl;

R⁴ is selected from hydrogen and C₁-C₁₀-alkyl;

or R³ and R⁴ form together with the carbon atoms bonded thereto a non-aromatic 5-membered ring unsubstituted or substituted by one or more of C₁-C₁₀-alkyl; or form together a non-aromatic 6-membered ring;

and X is selected from N and N-oxide;

and pharmaceutically acceptable salts thereof.

In another embodiment, the present invention provides a compound as above with the exception of 1,3-dibenzylpteridine-2,4(1H,3H)-dione.

In another embodiment:

R¹ is selected from benzyl substituted by chloro;

R² is selected from propyl; allyl; 3,3-dimethylbutyl; 2-oxo-3,3-dimethylbutyl; 2-hydroxy-3,3,3-trifluoropropyl; 2-trimethylsilylethyl; cyclohexylmethyl; benzyl unsubstituted or substituted by one or more of chloro, methoxy, cyano, and benzoyl; 2-phenylethyl; and 2-(1H-pyrrol-1-yl)ethyl;

R³ is selected from hydrogen, methyl, ethyl, and tert-butyl;

R⁴ is selected from hydrogen, methyl, ethyl, and tert-butyl;

or R³ and R⁴ form together with the carbon atoms bonded thereto a non-aromatic 5-membered ring unsubstituted or substituted by one or more of methyl; or form together a non-aromatic 6-membered ring.

In another embodiment, the present invention relates to the compounds as denoted in Examples 1-23.

The general terms used in the definition of formula (I) have the following meanings:

C₁-C₁₀ alkyl is a straight or branched alkyl group, having from 1 to 10 carbon atoms, for example methyl, ethyl, n-propyl, isopropyl, n-butyl, isobutyl, secondary butyl, tertiary butyl, pentyl, isopentyl, hexyl or heptyl.

C₂-C₁₀ alkenyl is a straight or branched alkenyl group, having 2 to 10 carbon atoms, for example vinyl, allyl, isopropenyl and 1-butenyl.

C₃-C₁₀ cycloalkyl is a cyclic alkyl, having 3 to 10 carbon atoms such as cyclopropyl, cyclobutyl, cyclopentyl, cyclohexyl and adamantyl.

C₁-C₁₀ alkoxy is an alkoxy group having 1 to 10 carbon atoms, for example methoxy, ethoxy, n-propoxy, n-butoxy, isopropoxy, isobutoxy, secondary butoxy, tertiary butoxy, pentoxy, hexoxy or a heptoxy group.

The term aryl is herein defined as an aromatic ring having from 6 to 14 carbon atoms including both single rings and polycyclic compounds, such as phenyl, benzyl or naphthyl.

The term aroyl is herein defined as an aryl group bonded to a carbonyl group, such as benzoyl.

The term heteroaryl is herein defined as an aromatic ring having 3 to 14 carbon atoms, including both single rings and polycyclic compounds in which one or several of the ring atoms is either oxygen, nitrogen or sulphur, such as pyrazolyl, benzothiadiazolyl, benzothiazolyl, thienyl, imidazolyl, isoxazolyl, pyridinyl and pyrrolyl.

Halogen as used herein is selected from chlorine, fluorine, bromine or iodine.

A non-aromatic ring means a ring that is not aromatic. For example, when R³ and R⁴ above form together with the carbon atoms bonded thereto a non-aromatic 5-membered ring, it means that this 5-membered ring does not contain any double bonds except for the ring fused to said 5-membered ring. For a 6-membered ring, the situation is quite analogous.

When two or more groups are used in connection with each other, it means that each group is substituted by the immediately preceding group. For instance, aryl-C₁-C₁₀ alkyl means a C₁-C₁₀ alkyl group substituted by an aryl group.

When a group is substituted by two or more further groups, these further groups need not be the same. For instance, in tri-C₁-C₁₀ alkylsilyl, the C₁-C₁₀ alkyl groups may be the same or different C₁-C₁₀ alkyl groups.

When the compounds of formula (I) have at least one asymmetric carbon atom, they can exist in several stereochemical forms. The present invention includes the mixture of isomers as well as the individual stereoisomers. The present invention further includes geometrical isomers, rotational isomers, enantiomers, racemates and diastereomers.

Where applicable, the compounds of formula (I) may be used in neutral form, e.g. as a carboxylic acid, or in the form of a salt, preferably a pharmaceutically acceptable salt such as the sodium, potassium, ammonium, calcium or magnesium salt of the compound at issue.

The compounds of formula (I) are useful as positive allosteric GBR (GABA_{B} receptor) modulators. A positive allosteric modulator of the GABA_{B} receptor is defined as a compound which makes the GABA_{B} receptor more sensitive to GABA and GABA_{B} receptor agonists by binding to the GABA_{B} receptor protein at a site different from that used by the endogenous ligand. The positive allosteric GBR modulator acts synergistically with an agonist and increases potency and/or intrinsic efficacy of the GABA_{B} receptor agonist. It has also been shown that positive allosteric modulators acting at the GABA_{B} receptor can produce an agonistic effect. Therefore, compounds of formula (I) can be effective as full or partial agonists.

The compounds may be used as a positive allosteric GABA_{B} receptor modulator. Also envisaged is a pharmaceutical composition comprising a compound above as an active ingredient and a pharmaceutically acceptable carrier or diluent.

A further aspect of the invention is a compound of the formula (I) above for use in therapy.

As a consequence of the GABA_{B} receptor becoming more sensitive to GABA_{B} receptor agonists upon the administration of a positive allosteric modulator, an increased inhibition of transient lower esophageal sphincter relaxations (TLESR) for a GABA_{B} agonist is observed. Consequently, the present invention is directed to the use of a positive allosteric GABA_{B} receptor modulator according to formula (I), optionally in combination with a GABA_{B} receptor agonist, for the preparation of a medicament for the inhibition of transient lower esophageal sphincter relaxations (TLESRs).

A further aspect of the invention is the use of a compound of formula (I), optionally in combination with a GABA_{B} receptor agonist, for the manufacture of a medicament for the prevention of reflux.

Also envisaged is a compound of formula (I) for use in the treatment of gastroesophageal reflux disease (GERD).

Also envisaged is a compound of formula (I) for use in the prevention of reflux.

Also envisaged is a compound of formula (I) for use in the inhibition of transient lower esophageal sphincter relaxations (TLESRs).

Also envisaged is a compound of formula (I) for us in the treatment of a functional gastrointestinal disorder. The functional gastrointestinal disorder could be e g functional dyspepsia.

Also envisaged is a compound of formula (I) for use in the treatment of irritable bowel syndrome (IBS). Said IBS could be e g constipation predominant IBS, diarrhea predominant IBS, or alternating bowel movement predominant IBS.

Still a further aspect of the invention is the use of a compound of formula (I), optionally in combination with a GABA_{B} receptor agonist, for the manufacture of a medicament for the treatment of gastroesophageal reflux disease (GERD).

Effective management of regurgitation in infants would be an important way of preventing, as well as curing lung disease due to aspiration of regurgitated gastric contents, and for managing failure to thrive, inter alia due to excessive loss of ingested nutrient. Thus, a further aspect of the invention is the use of a compound of formula (I), optionally in combination with a GABA_{B} receptor agonist, for the manufacture of a medicament for the treatment of lung disease.

Another aspect of the invention is the use of a compound of formula (I), optionally in combination with a GABA_{B} receptor agonist, for the manufacture of a medicament for the management of failure to thrive.

Another aspect of the invention is the use of a compound of formula (I), optionally in combination with a GABA_{B} receptor agonist, for the manufacture of a medicament for the treatment or prevention of asthma, such as reflux-related asthma.

A further aspect of the invention is the use of a compound of formula (I), optionally in combination with a GABA_{B} receptor agonist, for the manufacture of a medicament for the treatment or prevention of laryngitis or chronic laryngitis.

A further aspect of the present invention is a method for the inhibition of transient lower esophageal sphincter relaxations (TLESRs), whereby a pharmaceutically and pharmacologically effective amount of a compound of formula (I), optionally in combination with a GABA_{B} receptor agonist, is administered to subject in need of such inhibition.

Another aspect of the invention is a method for the prevention of reflux, whereby a pharmaceutically and pharmacologically effective amount of a compound of formula (I), optionally in combination with a GABA_{B} receptor agonist, is administered to a subject in need of such prevention.

Still a further aspect of the invention is a method for the treatment of gastroesophageal reflux disease (GERD), whereby a pharmaceutically and pharmacologically effective amount of a compound of formula (I), optionally in combination with a GABA_{B} receptor agonist, is administered to a subject in need of such treatment.

Another aspect of the present invention is a method for the treatment or prevention of regurgitation, whereby a pharmaceutically and pharmacologically effective amount of a compound of formula (I), optionally in combination with a GABA_{B} receptor agonist, is administered to a subject in need of such treatment.

Yet another aspect of the invention is a method for the treatment or prevention of regurgitation in infants, whereby a pharmaceutically and pharmacologically effective amount of a compound of formula (I), optionally in combination with a GABA_{B} receptor agonist, is administered to a subject in need of such treatment.

Still a further aspect of the invention is a method for the treatment, prevention or inhibition of lung disease, whereby a pharmaceutically and pharmacologically effective amount of a compound of formula (I), optionally in combination with a GABA_{B} receptor agonist, is administered to a subject in need of such treatment. The lung disease to be treated may inter alia be due to aspiration of regurgitated gastric contents.

Still a further aspect of the invention is a method for the management of failure to thrive, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of asthma, such as reflux-related asthma, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of laryngitis or chronic laryngitis, whereby a pharmaceutically and pharmacologically effective amount of a compound of formula (I), optionally in combination with a GABA_{B} receptor agonist, is administered to a subject in need of such treatment.

A further embodiment is the use of a compound of formula (I), optionally in combination with a GABA_{B} receptor agonist, for the manufacture of a medicament for the treatment of a functional gastrointestinal disorder (FGD). Another aspect of the invention is a method for the treatment of a functional gastrointestinal disorder, whereby an effective amount of a compound of formula (I), optionally in combination with a GABA_{B} receptor agonist, is administered to a subject suffering from said condition.

A further embodiment is the use of a compound of formula (I), optionally in combination with a GABA_{B} receptor agonist, for the manufacture of a medicament for the treatment of functional dyspepsia. Another aspect of the invention is a method for the treatment of functional dyspepsia, whereby an effective amount of a compound of formula (I), optionally in combination with a GABA_{B} receptor agonist, is administered to a subject suffering from said condition.

Functional dyspepsia refers to pain or discomfort centered in the upper abdomen. Discomfort may be characterized by or combined with upper abdominal fullness, early satiety, bloating or nausea. Etiologically, patients with functional dyspepsia can be divided into two groups:
1—Those with an identifiable pathophysiological or microbiologic abnormality of uncertain clinical relevance (e.g. Helicobacter pylori gastritis, histological duodenitis, gallstones, visceral hypersensitivity, gastroduodenal dysmotility)
2—Patients with no identifiable explanation for the symptoms.

Functional dyspepsia can be diagnosed according to the following:

At least 12 weeks, which need not be consecutive within the preceding 12 months of
1—Persistent or recurrent dyspepsia (pain or discomfort centered in the upper abdomen) and
2—No evidence of organic disease (including at upper endoscopy) that is likely to explain the symptoms and
3—No evidence that dyspepsia is exclusively relieved by defecation or associated with the onset of a change in stool frequency or form.

Functional dyspepsia can be divided into subsets based on distinctive symptom patterns, such as ulcer-like dyspepsia, dysmotility-like dyspepsia and unspecified (non-specific) dyspepsia.

Currently existing therapy of functional dyspepsia is largely empirical and directed towards relief of prominent symptoms. The most commonly used therapies still include antidepressants.

A further aspect of the invention is the use of a compound according to formula (I), optionally in combination with a GABA_{B} receptor agonist, for the manufacture of a medicament for the treatment or prevention of irritable bowel syndrome (IBS), such as constipation predominant IBS, diarrhea predominant IBS or alternating bowel movement predominant IBS.

A further aspect of the invention is a method for the treatment or prevention of irritable bowel syndrome (IBS), whereby a pharmaceutically and pharmacologically effective amount of a compound of formula (I), optionally in combination with a GABA_{B} receptor agonist, is administered to a subject in need of such treatment.

IBS is herein defined as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern:
1—diarrhea predominant
2—constipation predominant
3—alternating bowel movements.

Abdominal pain or discomfort is the hallmark of IBS and is present in the three subgroups.

IBS symptoms have been categorized according to the Rome criteria and subsequently modified to the Rome II criteria. This conformity in describing the symptoms of IBS has helped to achieve consensus in designing and evaluating IBS clinical studies.

The Rome II diagnostic criteria are:
1—Presence of abdominal pain or discomfort for at least 12 weeks (not necessarily consecutively) out of the preceding year
2—Two or more of the following symptoms:
   a) Relief with defecation
   b) Onset associated with change in stool frequency
   c) Onset associated with change in stool consistency

A further aspect of the invention is the use of a compound according to formula (I), optionally in combination with a GABA_{B} receptor agonist, for the manufacture of a medicament for the treatment or prevention CNS disorders, such as anxiety.

A further aspect of the invention is a method for the treatment or prevention of CNS disorders, such as anxiety, whereby a pharmaceutically and pharmacologically effective amount of a compound of formula (I), optionally in combination with a GABA_{B} receptor agonist, is administered to a subject in need of such treatment.

A further aspect of the invention is the use of a compound according to formula (I), optionally in combination with a GABA_{B} receptor agonist, for the manufacture of a medicament for the treatment or prevention of depression.

A further aspect of the invention is a method for the treatment or prevention of depression, whereby a pharmaceutically and pharmacologically effective amount of a compound of formula (I), optionally in combination with a GABA_{B} receptor agonist, is administered to a subject in need of such treatment.

For the purpose of this invention, the term “agonist” should be understood as including full agonists as well as partial agonists, whereby a “partial agonist” should be understood as a compound capable of partially, but not fully, activating GABA_{B} receptors.

The wording “TLESR”, transient lower esophageal sphincter relaxations, is herein defined in accordance with Mittal, R. K., Holloway, R. H., Penagini, R., Blackshaw, L. A., Dent, J, 1995; Transient lower esophageal sphincter relaxation. Gastroenterology 109, pp. 601-610.

The wording “reflux” is defined as fluid from the stomach being able to pass into the esophagus, since the mechanical barrier is temporarily lost at such times.

The wording “GERD”, gastroesophageal reflux disease, is defined in accordance with van Heerwarden, M. A., Smout A. J. P. M, 2000; Diagnosis of reflux disease. Baillière's Clin. Gastroenterol. 14, pp. 759-774.

A “combination” according to the invention may be present as a “fix combination” or as a “kit of parts combination”.

A “fix combination” is defined as a combination wherein (i) a compound of formula (I); and (ii) a GABA_{B} receptor agonist are present in one unit. One example of a “fix combination” is a pharmaceutical composition wherein (i) a compound of formula (I) and (ii) a GABA_{B} receptor agonist are present in admixture. Another example of a “fix combination” is a pharmaceutical composition wherein (i) a compound of formula (I) and (ii) a GABA_{B} receptor agonist; are present in one unit without being in admixture.

A “kit of parts combination” is defined as a combination wherein (i) a compound of formula (I) and (ii) a GABA_{B} receptor agonist are present in more than one unit. One example of a “kit of parts combination” is a combination wherein (i) a compound of formula (I) and (ii) a GABA_{B} receptor agonist are present separately. The components of the “kit of parts combination” may be administered simultaneously, sequentially or separately, i.e. separately or together.

The term “positive allosteric modulator” is defined as a compound which makes a receptor more sensitive to receptor agonists by binding to the receptor protein at a site different from that used by the endogenous ligand.

The term “therapy” and the term “treatment” also include “prophylaxis” and/or prevention unless stated otherwise. The terms “therapeutic” and “therapeutically” should be construed accordingly.

Pharmaceutical Formulations

The compound of formula (I) can be formulated alone or in combination with a GABA_{B} receptor agonist.

For clinical use, the compound of formula (I), optionally in combination with a GABA_{B} receptor agonist, is in accordance with the present invention suitably formulated into pharmaceutical formulations for oral administration. Also rectal, parenteral or any other route of administration may be contemplated to the skilled man in the art of formulations. Thus, the compound of formula (I), optionally in combination with a GABA_{B} receptor agonist, is formulated with a pharmaceutically and pharmacologically acceptable carrier or adjuvant. The carrier may be in the form of a solid, semi-solid or liquid diluent.

In the preparation of oral pharmaceutical formulations in accordance with the invention, the compound of formula (I), optionally in combination with a GABA_{B} receptor agonist, to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of a compound of formula (I), optionally in combination with a GABA_{B} receptor agonist, with vegetable oil, fat, or other suitable vehicle for soft gelatine capsules. Hard gelatine capsules may contain a compound of formula (I), optionally in combination with a GABA_{B} receptor agonist, in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains a compound of formula (I), optionally in combination with a GABA_{B} receptor agonist, in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing a compound of formula (I), optionally in combination with a GABA_{B} receptor agonist, and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of formula (I), optionally in combination with a GABA_{B} receptor agonist, in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, a compound of formula (I), optionally in combination with a GABA_{B} receptor agonist, may be administered once or twice daily, depending on the severity of the patient's condition. A typical daily dose of the compounds of formula (I) is from 0.1 to 100 mg per kg body weight of the subject to be treated, but this will depend on various factors such as the route of administration, the age and weight of the patient as well as of the severity of the patient's condition.

Methods of Preparation

Hereinbelow, Schemes 1-2 denote methods for preparation of the compounds according to the present invention.

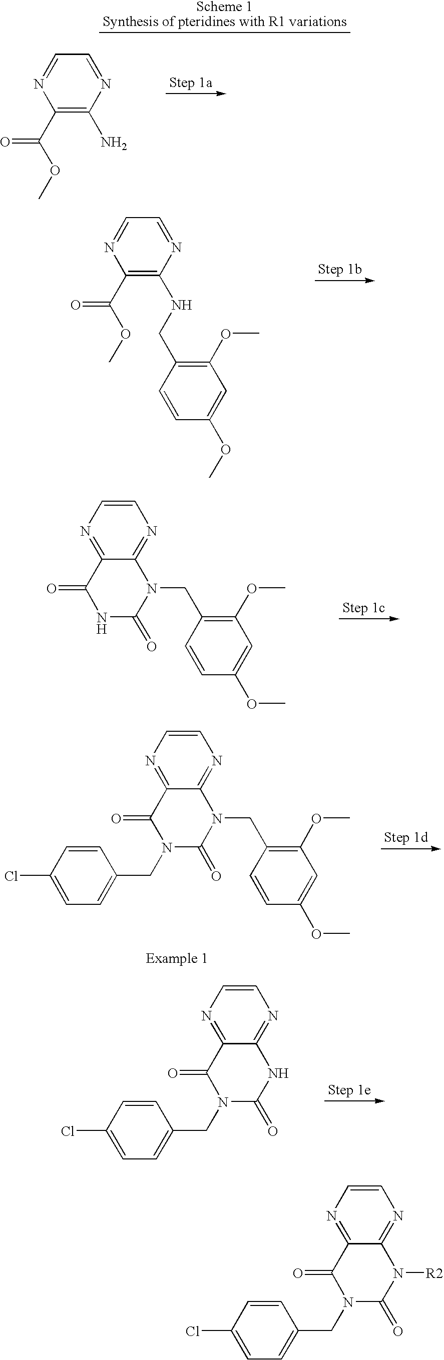

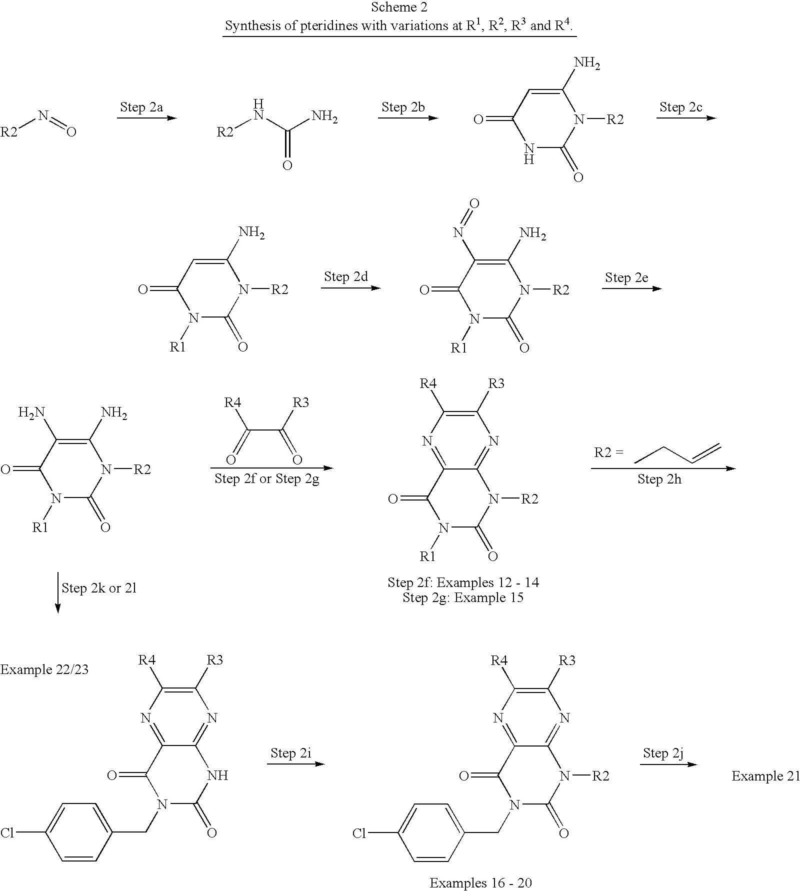

### EXAMPLES

### Abbreviations

DCM dichloromethane

DIPEA N,N-diisopropylethylamine

DMF N,N′-dimethylformamide

DMSO dimethylsulfoxide

EtOAc ethyl acetate

EtOH ethanol

FA formic acid

HPFC high performance flash chromatography

HPLC high performance liquid chromatography

LC-MS liquid chromatography mass spectroscopy

MeCN acetonitrile

MeOH methanol

NaOMe sodium methoxide

NMR nuclear magnetic resonance

TEA triethylamine

Tert tertiary

TFA trifluoroacetic acid

TMS trimethylsilyl

UV ultra violet

atm atmosphere

rt room temperature

h hour(s)

min minutes

br broad

s singlet

d doublet

t triplet

q quartet

m multiplet

sep septett

dd double doublet

td triple doublet

General Experimental Procedures

Phase Separator from IST was used. Flash column chromatography employed normal phase silica gel 60 (0.040-0.063 mm, Merck) or IST Isolute® SPE columns normal phase silica gel or Biotage Horizon™ HPFC System using silica FLASH+™ HPFC™ Cartridges. HPLC purifications were performed on either a Gilson preparative HPLC system with gradient pump system 333/334, GX-281 injector, UV/VIS detector 155. Trilution LC v.1.4 software. In acidic system equipped with an Kromasil C8 10 μm 250×20 ID mm column or Kromasil C8 10 μm 250×50 ID mm column and as gradient: mobile phase (buffer): H₂O/MeCN/FA 95/5/0.2 and mobile phase (organic): MeCN. In neutral system equipped with an Kromasil C8 10 μm 250×20 ID mm column or Kromasil C8 10 μm 250×50 ID mm column and as gradient: mobile phase (buffer): MeCN/0,1M NH₄OAc 5/95 and mobile phase (organic): MeCN. In basic system system equipped with an XBridge C18 10 μm 250×8 ID mm column or XBridge C18 10 μm 250×50 ID mm column and as gradient: mobile phase (buffer): H₂O/MeCN/NH₃ 95/5/0.2 and mobile phase (organic): MeCN. Or on a Waters preparative HPLC system equipped with a Kromasil C8 10 mm 250 mm×21.2 mm column and a gradient mobile phase (buffer): MeCN/0,1M NH₄OAc 5/95 and mobile phase (organic): MeCN or on a Waters FractionLynx HPLC system with a mass triggered fraction collector, equipped with a Xbridge Prep C18 5μ 19 mm×150 mm column using MeCN/NH₃ buffer system with a gradient from 95% mobile phase A (0.2% NH3 in water, pH10) to 95% mobile phase B (100% MeCN) unless otherwise stated. ¹H NMR and ¹³C NMR measurements were performed on a BRUKER ACP 300 or on a Varian Inova 400, 500 or 600 spectrometer, operating at ¹H frequencies of 300, 400, 500, 600 MHz, respectively, and ¹³C frequencies of 75, 100, 125 and 150 MHz, respectively. Chemical shifts are given in δ values (ppm) with the solvents used as internal standard, unless otherwise stated. Microwave heating was performed using single node heating in a Smith Creator or Emrys Optimizer from Personal Chemistry, Uppsala, Sweden. Mass spectral data were obtained using a Micromass LCT or Waters Q-Tof micro system and, where appropriate, either positive ion data or negative ion data were collected.

Compound names generated by ACD/Name Release 9.0. Product Version: 9.04 (Build 6210, 20 Jul. 2005).

Explanation to Plate-NMR:

*The solutions are taken from a concentrated sample dissolved in (CH₃)₂SO and are diluted with (CD₃)₂SO. Since a substantial amount of (CH₃)₂SO is present in the sample, first a pre-scan is run and analysed to automatically suppress the (CH₃)₂SO (2.54 ppm) and H₂O (3.3 ppm) peaks. This means that in this so-called wet1D experiment the intensity of peaks that reside in these areas around 3.3 ppm and 2.54 ppm are reduced. Furthermore impurities are seen in the spectrum which give rise to a triplet at 1.12 ppm, a singlet at 2.96 ppm and two multiplets between 2.76-2.70 ppm and 2.61-2.55 ppm. Most probably these impurities are dimethylsulfone and diethylsulfoxide.

### Example 1

### 3-(4-chlorobenzyl)-1-(2,4-dimethoxybenzyl)pteridine-2,4(1H,3H)-dione was Synthesized According to Scheme 1

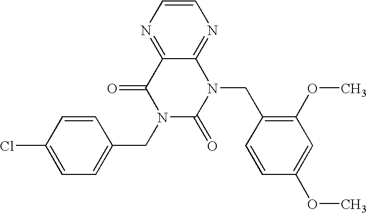

### Step 1a: Methyl 3-[(2,4-dimethoxybenzyl)amino]pyrazine-2-carboxylate

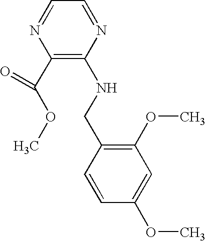

Sodium triacetoxyborohydride (7.82 g, 36.89 mmol) was added to a mixture of methyl 3-aminopyrazine-2-carboxylate (4.00 g, 26.12 mmol) and 2,4-dimethoxybenzaldehyde (4.83 g, 29.04 mmol) in 1,2-dichloroethane (90 ml). The reaction was continued at room temperature for 24 hours. Sodium triacetoxyborohydride (13.0 g, 61.34 mmol) and 2,4-dimethoxybenzaldehyde (8.0 g, 48.14 mmol) were added and the reaction continued at room temperature over night. Water and DCM were added and the phases separated. The product was purified further by flash chromatography (SiO₂, heptane:ethyl acetate, product came at 50% ethyl acetate) and recrystallisation (DCM/heptane) to give a slightly yellow solid (5.00 g, 63%).

¹H NMR (400 MHz, CDCl₃) δ 8.35-8.27 (br, 1H), 8.22 (d, 1H), 7.82 (d, 1H), 7.20 (d, 1H), 6.48-6.43 (m, 1H), 6.43-6.37 (m, 1H), 4.63 (d, 2H), 3.93 (s, 3H), 3.84 (s, 3H), 3.77 (s, 3H).

MS m/z 304 (M+H)⁺.

### Step 1b: 1-(2,4-dimethoxybenzyl)pteridine-2,4(1H,3H)-dione

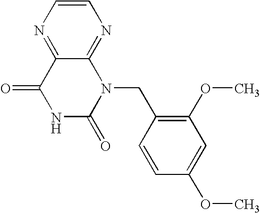

Trichloroacetyl isocyanate (1.1 ml, 9.23 mmol) was added to a solution of methyl 3-[(2,4-dimethoxybenzyl)amino]pyrazine-2-carboxylate (2.55 g, 8.41 mmol, ref example 1 step 1a product) in DCM (50 ml). The reaction was continued at room temperature for 16 hours. The solvent was evaporated. A solution of sodium methoxide (3.3 g, 61.09 mmol) in methanol (50 ml) was added. The mixture was heated to 60° C. for 3 hours. Water and DCM were added, the phases separated and the organic phase dried (2.70 g, crude).

¹H NMR (400 MHz, CDCl₃) δ 9.60-9.00 (br, 1H), 8.57 (s, 1H), 8.54 (s, 1H), 6.91 (d, 1H). 6.41 (s, 1H), 6.33 (d, 1H), 5.41 (s, 2H), 3.76 (s, 3H), 3.73 (s, 3H).

MS m/z 315 (M+H)⁺.

### Step 1c

### Example 1

### 3-(4-chlorobenzyl)-1-(2,4-dimethoxybenzyl)pteridine-2,4(1H,3H)-dione

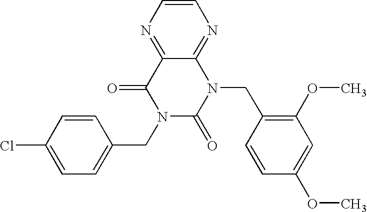

A solution of 4-chlorobenzyl bromide (1.28 g, 6.22 mmol) in DMF (5 ml) was added to a mixture of 1-(2,4-dimethoxybenzyl)pteridine-2,4(1H,3H)-dione (1.77 g, crude, ref. example 1 step 1b product) and potassium carbonate (1.17 g, 8.49 mmol) in DMF (15 ml). The reaction was continued at room temperature for 65 hours. Water and DCM were added and the phases separated (2.43 g, crude). 41 mg of the crude was purified further by preparatory HPLC (Xbridge C18 column, NH₃ (aq, 0.2%):MeCN) to give an almost white powder (12 mg, 29% for two steps).

¹H NMR (600 MHz, wet DMSO) δ 8.68 (d, 1H), 8.60 (d, 1H), 7.39 (d, 2H), 7.35 (d, 2H), 6.85 (d, 1H), 6.54 (d, 1H), 6.29 (dd, 1H), 5.23 (s, 2H), 5.12 (s, 2H), 3.77 (s, 3H), 3.68 (s, 3H).

HRMS Calcd for [C₂₂H₁₉ClN₄O₄+H]+: 439.1173. Found: 439.1164.

### Example 2

### 3-(4-chlorobenzyl)-1-propylpteridine-2,4(1H,3H)-dione was Synthesized According to Scheme 1

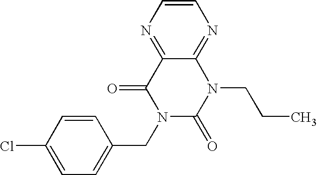

### Step 1d: 3-(4-chlorobenzyl)pteridine-2,4(1H,3H)-dione

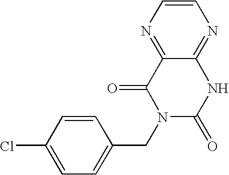

A solution of 3-(4-chlorobenzyl)-1-(2,4-dimethoxybenzyl)pteridine-2,4(1H,3H)-dione (2.41 g, crude, ref example 1) in TFAA (30% in 1,2-dichloroethane, 15 ml) was heated to 100° C. for 50 hours. The solvent was evaporated and the mixture suspended in methanol. Water was added and the product collected by filtration. Washed with water and dried at reduced pressure (2.10 g, crude).

¹H NMR (400 MHz, DMSO) δ 8.80 (s, 1H), 8.69 (s, 1H), 7.54-7.46 (m, 4H), 5.19 (s, 2H).

MS m/z 289, 291 (M+H)⁺.

### Step 1e

### Example 2

### 3-(4-chlorobenzyl)-1-propylpteridine-2,4(1H,3H)-dione

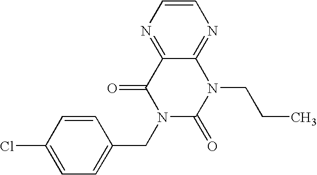

A mixture of 3-(4-chlorobenzyl)pteridine-2,4(1H,3H)-dione (29 mg, crude, ref. example 2 step 1d product), propylbromide (62 mg, 0.50 mmol) and potassium carbonate (31 mg, 0.22 mmol) in DMF (1 ml) was stirred for 24 hours. Water and DCM were added and the phases separated. The product was purified further by preparatory HPLC (Xbridge C18 column, NH₃ (aq, 0.2%):MeCN) to give an almost white powder (11 mg, 45% for four steps).

¹H NMR (600 MHz, wet DMSO) δ 8.77 (d, 1H), 8.59 (d, 1H), 7.39-7.33 (m, 4H), 5.10 (s, 2H), 4.14-4.10 (m, 2H), 1.67-1.59 (m, 2H), 0.88 (t, 3H).

HRMS Calcd for [C₁₆H₁₅ClN₄O₂+H]+: 331.0962. Found: 331.0963.

Examples 3-11 were Prepared According to Example 2:

### Example 3

### 3-(4-chlorobenzyl)-1-[2-(trimethylsilyl)ethyl]pteridine-2,4(1H,3H)-dione

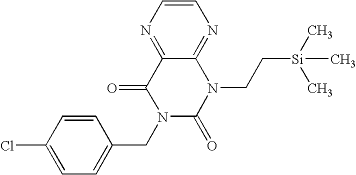

From 3-(4-chlorobenzyl)pteridine-2,4(1H,3H)-dione (200 mg, crude, ref. example 2 step 1d product), 107 mg (53% for four steps) of the title compound was isolated

¹H NMR (400 MHz, CDCl₃) δ 8.53 (d, 1H), 8.46 (d, 1H), 7.40 (d, 2H), 7.16 (d, 2H), 5.15 (s, 2H), 4.29-4.20 (m, 2H), 0.96-0.88 (m, 2H), 0.00 (s, 9H).

HRMS Calcd for [C₁₈H₂₁ClN₄O₂Si+H]+: 389.1201. Found: 389.1198.

### Example 4

### 3-(4-chlorobenzyl)-1-(cyclohexylmethyl)pteridine-2,4(1H,3H)-dione

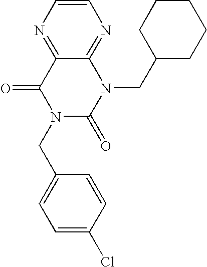

From 3-(4-chlorobenzyl)pteridine-2,4(1H,3H)-dione (29 mg, crude, ref. example 2 step 1d product), 9.7 mg (33% for four steps) of the title compound was isolated.

¹H NMR (600 MHz, wet DMSO) δ 8.76 (d, 1H), 8.58 (d, 1H), 7.39-7.31 (m, 4H), 5.10 (s, 2H), 4.03 (d, 2H), 1.85-1.75 (m, 1H), 1.65-1.50 (m, 4H), 1.15-1.03 (m, 4H), 1.02-0.92 (m, 2H).

HRMS Calcd for [C₂₀H₂₁ClN₄O₂+H]+: 385.1431. Found: 385.1433.

### Example 5

### 3-(4-chlorobenzyl)-1-(3,3-dimethyl-2-oxobutyl)pteridine-2,4(1H,3H)-dione

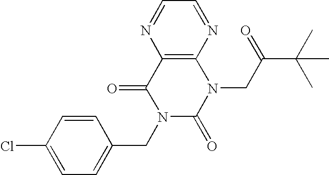

From 3-(4-chlorobenzyl)pteridine-2,4(1H,3H)-dione (29 mg, crude, ref. example 2 step 1d product), 11.2 mg (38% for four steps) of the title compound was isolated.

¹H NMR (600 MHz, wet DMSO) δ 8.72 (d, 1H), 8.64 (d, 1H), 7.38-7.33 (m, 4H), 5.26 (s, 2H), 5.11 (s, 2H), 1.20 (s, 9H).

HRMS Calcd for [C₁₉H₁₉ClN₄O₃+H]+: 387.1224. Found: 387.1213.

### Example 6

### 3-(4-chlorobenzyl)-1-(3,3-dimethylbutyl)pteridine-2,4(1H,3H)-dione

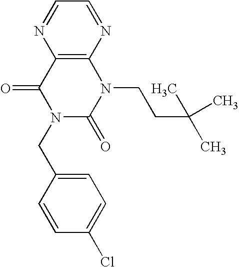

From 3-(4-chlorobenzyl)pteridine-2,4(1H,3H)-dione (29 mg, crude, ref. example 2 step 1d product), 12.4 mg (44% for four steps) of the title compound was isolated.

¹H NMR (600 MHz, wet DMSO) δ 8.77 (d, 1H), 8.59 (d, 1H), 7.39-7.33 (m, 4H), 5.09 (s, 2H), 4.20-4.16 (m, 2H), 1.52-1.47 (m, 2H), 0.95 (s, 9H).

HRMS Calcd for [C₁₉H₂₁ClN₄O₂+H]+: 373.1431. Found: 373.1435.

### Example 7

### 1-benzyl-3-(4-chlorobenzyl)pteridine-2,4(1H,3H)-dione

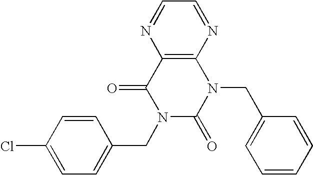

From 3-(4-chlorobenzyl)pteridine-2,4(1H,3H)-dione (29 mg, crude, ref. example 2 step 1d product) 14.3 mg (50% for four steps) of the title compound was isolated.

¹H NMR (600 MHz, wet DMSO) δ 8.73 (d, 1H) 8.61 (d, 1H), 7.41-7.18 (m, 9H), 5.37 (s, 2H), 5.12 (s, 2H).

HRMS Calcd for [C₂₀H₁₅ClN₄O₂+H]+: 379.0962. Found: 379.0973.

### Example 8

### 1,3-bis(4-chlorobenzyl)pteridine-2,4(1H,3H)-dione

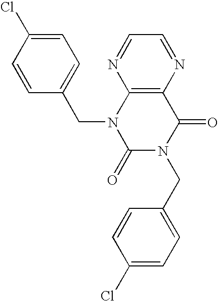

From 3-(4-chlorobenzyl)pteridine-2,4(1H,3H)-dione (29 mg, crude, ref. example 2 step 1d product), 16 mg (51% for four steps) of the title compound was isolated.

¹H NMR (600 MHz, wet DMSO) δ 8.72 (d, 1H), 1.62 (d, 1H), 7.41-7.30 (m, 8H), 5.35 (s, 2H), 5.11 (s, 2H).

HRMS Calcd for [C₂₀H₁₄Cl₂N₄O₂+H]+: 413.0572. Found: 413.0573.

### Example 9

### 3-(4-chlorobenzyl)-1-(4-methoxybenzyl)pteridine-2,4(1H,3H)-dione

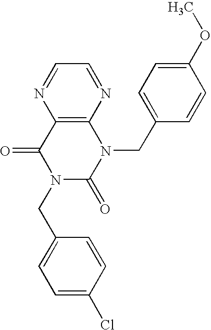

From 3-(4-chlorobenzyl)pteridine-2,4(1H,3H)-dione (29 mg, crude, ref. example 2 step 1d product), 16.2 mg (52% for four steps) of the title compound was isolated.

¹H NMR (600 MHz, wet DMSO) δ 8.75 (d, 1H), 8.61 (d, 1H), 7.40-7.37 (m, 2H), 7.36-7.33 (m, 2H), 7.30-7.26 (m, 2H), 6.83-6.79 (m, 2H), 5.30 (s, 2H), 5.11 (s, 2H), 3.67 (s, 3H).

HRMS Calcd for [C₂₁H₁₇ClN₄O₃+H]+: 409.1067. Found: 409.1073.

### Example 10

### 1-(4-benzoylbenzyl)-3-(4-chlorobenzyl)pteridine-2,4(1H,3H)-dione

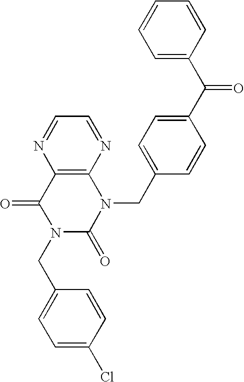

From 3-(4-chlorobenzyl)pteridine-2,4(1H,3H)-dione (29 mg, crude, ref. example 2 step 1d product), 16.6 mg (45% for four steps) of the title compound was isolated.

¹H NMR (600 MHz, wet DMSO) δ 8.73 (d, 1H), 8.63 (d, 1H), 7.70-7.62 (m, 5H), 7.54-7.49 (m, 4H), 7.42-7.39 (m, 2H), 7.37-7.33 (m, 2H), 5.48 (s, 2H), 5.13 (s, 2H).

HRMS Calcd for [C₂₇H₁₉ClN₄O₃+H]+: 483.1224. Found: 483.1194.

### Example 11

### 3-(4-chlorobenzyl)-1-(2-phenylethyl)pteridine-2,4(1H,3H)-dione

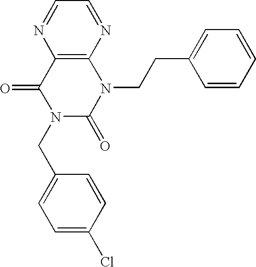

From 3-(4-chlorobenzyl)pteridine-2,4(1H,3H)-dione (29 mg, crude, ref. example 2 step 1d product), 13 mg (44% for four steps) of the title compound was isolated.

¹H NMR (600 MHz, wet DMSO) δ 8.78 (d, 1H), 8.61 (d, 1H), 7.36-7.30 (m, 4H), 7.26-7.22 (m, 2H), 7.21-7.15 (m, 3H), 5.09 (s, 2H), 4.41-4.37 (m, 2H), 2.94-2.89 (m, 2H).

HRMS Calcd for [C₂₁H₁₇ClN₄O₂+H]+: 393.1118. Found: 393.1112.

### Example 12

### 3-(4-chlorobenzyl)-6,8-dimethyl-1-propyl-7,8-dihydro-1H-cyclopenta[g]pteridine-2,4(3H,6H)-dione, was Synthesized According to Scheme 2

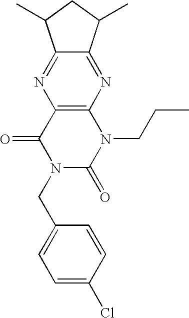

### Step 2a: Synthesis of 1-propylurea

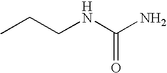

To 100 mL of a 2 M solution of ammonia in ethanol (200 mmol) was added 7 mL (73.7 mmol) propylisocyanate. The resulting mixture was stirred at room temperature for 30 min, then the solvents were evaporated. The residue was dried to give 7.44 g (72.8 mmol, 99%) of 1-propylurea as a colorless solid that was used without further purification.

¹H NMR (400 MHz, DMSO-d₆) δ 5.86 (s, 1H), 5.31 (s, 2H), 2.83-2.89 (m, 2H), 1.26-1.36 (m, 2H), 0.78 (t, 7.5 Hz, 3H).

¹³C NMR (100 MHz, DMSO-d₆) δ 159.4, 41.7, 23.8, 12.0.

### Step 2b: Synthesis of 6-amino-1-propylpyrimidine-2,4(1H,3H)-dione

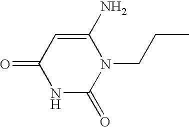

To a mixture of 26.86 g (263 mmol) of 1-propylurea, ref example 12 step 2a product, and 30.74 g (271 mmol) of ethyl cyanoacetate was added 150 mL of a 21% solution of sodium ethoxide in ethanol. The resulting mixture was heated under reflux with exclusion of moisture (drying tube on reflux condenser) overnight. The reaction mixture was cooled to room temperature and the solvents evaporated. The residue was dissolved in 200 mL water and heated for 2 h under reflux. After cooling to room temperature, conc. HCl was added slowly until pH=4. A precipitate formed. The resulting suspension was stirred at room temperature overnight, then the solid was collected, washed with water and dried. 23.23 g (137.3 mmol, 52%) of 6-amino-1-propylpyrimidine-2,4(1H,3H)-dione was isolated as a beige solid that was used without further purification.

¹H NMR (400 MHz, DMSO-d₆) δ 10.24 (s, 1H), 6.73 (s, 2H), 4.48 (s, 1H), 3.29-3.66 (m, 2H), 1.42-1.52 (m, 2H), 0.81 (t, 7.5 Hz, 3H).

### Step 2c: Synthesis of 6-amino-3-(4-chlorobenzyl)-1-propylpyrimidine-2,4(1H,3H)-dione

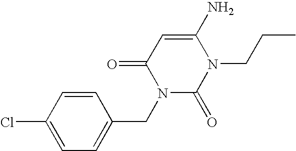

8.46 g (50 mmol) of 6-amino-1-propylpyrimidine-2,4(1H,3H)-dione, ref example 12 step 2b product, was suspended in 20 mL DMF, then 6.28 g (52.7 mmol) of dimethylformamide dimethylacetal was added and the resulting mixture warmed to 40° C. for 3 h, then addition of 10 mL DMF and 450 mg (3.8 mmol) of dimethylformamide dimethylacetal. Stirring was continued at 40° C. for additional 30 min, then 12.33 g (60 mmol) of 4-chlorobenzyl bromide and 13.82 g (100 mmol) of K₂CO₃ were added in one portion followed by 10 mL DMF. The reaction temperature was increased to 80° C. and after 1 h additional 3.08 g (15 mmol) of 4-chlorobenzyl bromide and 15 mL DMF were added. The reaction mixture was stirred for three days at 80° C., then cooled to room temperature. 150 mL ethyl acetate was added to the reaction mixture and then filtered. The solids were washed with ethyl acetate. The combined filtrated were evaporated. The solid residue was suspended in methanol and sonicated until a fine suspension resulted. The solid was collected and washed with methanol. From the combined filtrates additional solid could be isolated using the same procedure. 6.94 g of colorless solid was isolated. To two portions (5.04 g and 1.9 g) of this solid was added 5 mL MeOH and 15 mL conc. aq. NH₃ each and the resulting mixtures were heated in sealed vials for 1 h to 120° C. using microwave heating. The two reaction mixtures were combined, the solvents evaporated and the residue crystallized from methanol/water. 4.93 g (16.78 mmol, 33%) of 6-amino-3-(4-chlorobenzyl)-1-propylpyrimidine-2,4(1H,3H)-dione was isolated as colorless solid.

¹H NMR (400 MHz, DMSO-d₆) δ 7.30 (d, 8.5 Hz, 2H), 7.20 (d, 8.5 Hz, 2H), 6.87 (s, 2H), 4.84 (s, 2H), 4.68 (s, 1H), 3.66-3.71 (m, 2H), 1.42-1.53 (m, 2H), 0.80 (t, 7.4 Hz, 3H).

¹³C NMR (100 MHz, DMSO-d₆) δ 161.7, 155.2, 152.0, 138.0, 132.0, 130.0, 128.8, 75.5, 44.0, 43.0, 21.4, 11.4.

MS m/z 294.1 (M+H)⁺

### Step 2d: Synthesis of 6-amino-3-(4-chlorobenzyl)-5-nitroso-1-propylpyrimidine-2,4(1H,3H)-dione

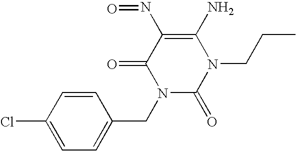

4.89 g (16.65 mmol) 6-amino-3-(4-chlorobenzyl)-1-propylpyrimidine-2,4(1H,3H)-dione, ref example 12 step 2c product, was dissolved in 75 mL acetic acid at 80° C. Then 2.297 g (33.29 mmol) NaNO₂ in 10 mL water was added dropwise. After ca. 1 min a thick purple slurry resulted. Heating was continued for 30 min, then addition of ca. 200 mL water and this mixture warmed to 80° C. for 30 min, then cooled in an ice-bath. The bright purple precipitate was collected and washed with water, then dried. 5.51 g (17.07 mmol, quant.) crude 6-amino-3-(4-chlorobenzyl)-5-nitroso-1-propylpyrimidine-2,4(1H,3H)-dione was isolated as pink-purple solid that was used without further purification.

¹H NMR (400 MHz, DMSO-d₆) δ 13.11 (s, 1H), 9.14 (s, 1H), 7.31-7.39 (m, 4H), 5.03 (s, 2H), 3.71-3.76 (m, 2H), 1.43-1.53 (m, 2H), 0.83 (t, 7.3 Hz, 3H).

¹³C NMR (100 MHz, DMSO-d₆) δ 160.7, 149.9, 146.2, 139.7, 136.7, 132.4, 130.2, 128.9, 44.1, 43.4, 20.3, 11.3.

MS m/z 323.2 (M+H)⁺

### Step 2e: Synthesis of 5,6-diamino-3-(4-chlorobenzyl)-1-propylpyrimidine-2,4(1H,3H)-dione

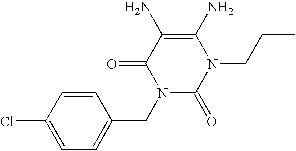

2.33 g (7.22 mmol) 6-amino-3-(4-chlorobenzyl)-5-nitroso-1-propylpyrimidine, 2,4(1H,3H)-dione, ref example 12 step 2d product, was suspended in acetonitrile, then 60 mL of ca. 13% aq. ammonia was added and an orange solution resulted. This solution was warmed to 80° C. in an oil-bath. Then 2.51 g (14.44 mmol) sodium dithionite was added as a solid in portions over 5 min. and the resulting mixture stirred at 80° C. for 1 h. The reaction mixture was cooled to room temperature, reduced to half its volume (evaporation of acetonitrile) and diluted with more water. The formed solid was collected, washed with water and dried. 1.9 g (6.15 mmol, 85%) 5,6-diamino-3-(4-chlorobenzyl)-1-propylpyrimidine-2,4(1H,3H)-dione was isolated as slightly green solid.

¹H NMR (400 MHz, DMSO-d₆) δ 7.27 (d, 8.3 Hz, 2H), 7.19 (d, 8.3 Hz, 2H), 6.23 (s, 2H), 4.88 (s, 2H), 3.70-3.75 (m, 2H), 2.86 (s, 2H), 1.44-1.50 (m, 2H), 0.78 (t, 7.5 Hz, 3H).

¹³C NMR (100 MHz, DMSO-d₆) δ 159.3, 150.2, 145.4, 137.9, 132.1, 130.1, 128.8, 96.3, 44.3, 43.5, 21.5, 11.4.

MS m/z 309.2 (M+H)⁺

### Step 2f

### Example 12

### 3-(4-chlorobenzyl)-6,8-dimethyl-1-propyl-7,8-dihydro-1H-cyclopenta[g]pteridine-2,4(3H,6H)-dione

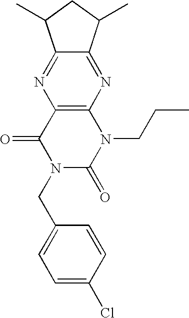

62 mg (0.2 mmol) 5,6-diamino-3-(4-chlorobenzyl)-1-propylpyrimidine-2,4(1H,3H)-dione, ref example 12 step 2e product, and 25 mg (0.2 mmol) 3,5-Dimethyl-cyclopentane-1,2-dione were dissolved in 1 mL methanol/acetic acid 9:1. The resulting mixture was heated to 160 C for 25 min using microwave heating. Then additional 5 mg (0.038 mmol) 3,5-Dimethyl-cyclopentane-1,2-dione was added. Heating continued for 15 min at 160 C. The solvents were evaporated, the residue dissolved in DCM and washed with sat. NaHCO₃. Organic layer filtered through a phase separator and evaporated. The residue was purified by reversed phase HPLC. 26 mg (0.065 mmol, 32%) of the title compound was isolated as a mixture of isomers.

¹H-NMR (600 MHz, (CH₃)₂SO*, (CD₃)₂SO)) δ 7.32-7.34 (m, 4H), 5.09 (s, 2H), 4.10-4.17 (m, 2H), 3.37-3.43 (m, 0.4H), 2.00-2.03 (m, 0.5H), 1.61-1.68 (m, 2H), 1.23-1.31 (m, 7.5H), 1.00-1.03 (m, 1.6H), 0.84-0.87 (m, 4H).

HRMS Calcd for [C₂₁H₂₃ClN₄O₂+H]⁺: 399.1588. Found: 399.1585.

Examples 13-15 were Prepared According to Example 12 Besides Described Exceptions:

### Example 13

### 3-(4-chlorobenzyl)-6,7-diethyl-1-propylpteridine-2,4(1H,3H)-dione

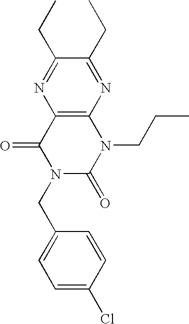

From 62 mg (0.2 mmol) 5,6-diamino-3-(4-chlorobenzyl)-1-propylpyrimidine-2,4(1H,3H)-dione, ref example 12 step 2e product, 76 mg (0.196 mmol, 98%) of the title compound was isolated as a colorless solid after flash column chromatography on silica using ethyl acetate/hexanes as eluent.

¹H-NMR (500 MHz, CDCl₃) δ 7.51 (d, 8.5 Hz, 2H), 7.24 (d, 8.5 Hz, 2H), 5.24 (s, 2H), 4.25-4.29 (m, 2H), 2.93-2.99 (m, 4H), 1.71-1.79 (m, 2H), 1.32-1.37 (m, 6H), 0.97 (t, 7.5 Hz, 3H).

HRMS Calcd for [C₂₀H₂₃ClN₄O₂+H]⁺: 387.1588. Found: 387.1575.

### Example 14

### 1-allyl-3-(4-chlorobenzyl)-6,7,8,9-tetrahydrobenzo[g]pteridine-2,4(1H,3H)-dione

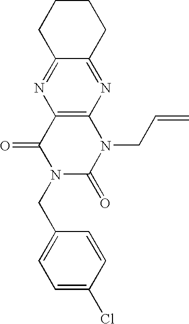

### Step 2b: 1-allyl-6-aminopyrimidine-2,4(1H,3H)-dione

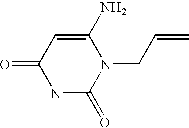

From n-propylurea (90 g, 0.8982 mol, ref. example 12 step 2a product) was the title compound isolated as a pale yellow solid (70 g, 46.5%).

### Step 2c: 1-allyl-6-amino-3-(4-chlorobenzyl)pyrimidine-2,4(1H,3H)-dione

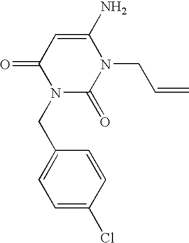

From 1-allyl-6-aminopyrimidine-2,4(1H,3H)-dione (40 g, 0.2395 mol, ref. example 13 step 2b product) was the title compound isolated as a white solid (30 g, 43%).

1H-NMR (400 MHz, CDCl₃) δ 7.39-7.32 (d, 2H), 7.25-7.19 (d, 2H), 6.17 (br, 2H), 5.94-5.81 (m, 1H), 5.32-5.19 (m, 2H), 5.02 (s, 2H), 4.99 (s, 2H), 4.61 (s, 1H),

MS 291.9 (M+H)+

### Step 2d: 1-allyl-6-amino-3-(4-chlorobenzyl)-5-nitrosopyrimidine-2,4(1H,3H)-dione

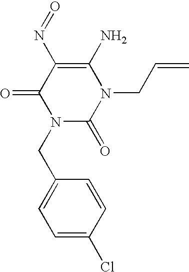

From 7.4 g (25.37 mmol) of 1-allyl-6-amino-3-(4-chlorobenzyl)pyrimidine-2,4(1H,3H)-dione, ref. example 13 step 2c product, using MeOH and acetic acid as solvent, 7.54 g (23.51 mmol, 93%) of the title compound was isolated as purple solid.

¹H-NMR (500 MHz, DMSO-d₆) δ 13.07 (br, 1H), 8.99 (br, 1H), 7.29-7.38 (m, 4H), 5.72-5.82 (m, 1H), 5.09-5.17 (m, 2H), 5.05 (s, 2H), 4.46-4.48 (m, 2H).

MS m/z 321.1 (M+H)⁺.

### Step 2e: 1-allyl-5,6-diamino-3-(4-chlorobenzyl)pyrimidine-2,4(1H,3H)-dione

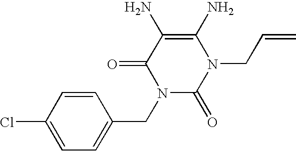

7.54 g (23.51 mmol) 1-allyl-6-amino-3-(4-chlorobenzyl)-5-nitrosopyrimidine-2,4(1H,3H)-dione, ref. example 13 step 2d product, was suspended in 50 mL conc. ammonia/water 1:1, then acetonitrile (ca. 30 mL) was added until an orange-colored solution resulted. The mixture was warmed to 75 C and 8.19 g (47 mmol) sodium dithionite was added in portions every 5 minutes until the orange color had faded. Under the course of the reaction more water was added to wash the walls of the flask. The reaction mixture was then stirred at 75 C for 1 h. Acetonitrile was evaporated, a precipitate occurred. The mixture was neutralized (pH ca. 7-8) by the addition of 1N HCl and cooled in an ice-bath for 30 min. The precipitate was collected, washed with water and dried. 6.51 g (21.2 mmol, 90%) of the title compound was isolated as a pale yellow solid.

¹H-NMR (400 MHz, DMSO-d₆) δ 7.31 (d, 8.5 Hz, 2H), 7.21 (d, 8.5 Hz, 2H), 6.17 (br, 2H), 5.74-5.84 (m, 1H), 4.98-5.09 (m, 2H), 4.91 (s, 2H), 4.46 (d, 4.6 Hz, 2H, 2H), 2.90 (br, 2H).

MS m/z 307.1 (M+H)⁺.

### Step 2f

### Example 14

### 1-allyl-3-(4-chlorobenzyl)-6,7,8,9-tetrahydrobenzo[g]pteridine-2,4(1H,3H)-dione

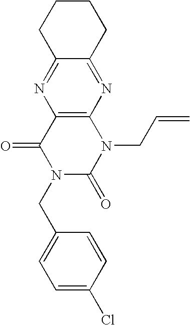

1.21 g (3.95 mmol) 1-allyl-5,6-diamino-3-(4-chlorobenzyl)pyrimidine-2,4(1H,3H)-dione, ref. example 13 step 2e product, and 442 mg (3.95 mmol) 1,2-cyclohexanedione were heated to 160 C for 5 min using microwave heating. The combined organic layers were dried over MgSO₄ and evaporated. The residue was purified by flash column chromatography on silica gel using 100% DCM, then DCM/EtOAc 9:1 as eluent. 1.05 g (2.74 mmol, 69%) of the title compound was isolated as yellowish solid.

¹H-NMR (500 MHz, CDCl₃) δ 7.48 (d, 8.5 Hz, 2H), 7.23 (d, 8.5 Hz, 2H), 5.85-5.96 (m, 1H), 5.16-5.28 (m, 4H), 4.86 (d, 5.9 Hz, 2H), 2.95-3.08 (m, 4H), 1.92-1.97 (m, 4H).

### Example 15

### 3-(4-chlorobenzyl)-6,7-dimethyl-1-propylpteridine-2,4(1H,3H)-dione, was Synthesized According to Scheme 2

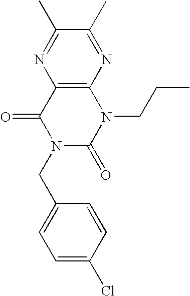

### Step 2g

### Example 15

### 3-(4-chlorobenzyl)-6,7-dimethyl-1-propylpteridine-2,4(1H,3H)-dione

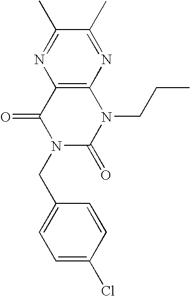

80 mg (0.26 mmol) 5,6-diamino-3-(4-chlorobenzyl)-1-propylpyrimidine-2,4(1H,3H)-dione, ref. example 12 step 2e product, and 31 mg (0.36 mmol) 2,3-butandione were dissolved in 1 mL dioxane. The resulting mixture was heated in a sealed vessel to 110 C for 1 h, then 100 C overnight. The solvents were evaporated, and the residue purified by reversed phase HPLC. 44 mg (0.067 mmol, 26%) of the ca. 55% pure title compound was isolated.

HRMS Calcd for [C₁₈H₁₉ClN₄O₂+H]⁺: 359.1275. Found: 359.1275.

### Example 16

### 3-{[3-(4-chlorobenzyl)-2,4-dioxo-3,4,6,7,8,9-hexahydrobenzo[g]pteridin-1(2H)-yl]methyl}benzonitrile was Synthesized According to Scheme 2

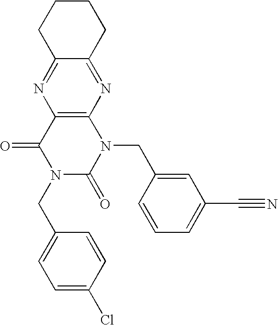

### Step 2h: 3-(4-chlorobenzyl)-6,7,8,9-tetrahydrobenzo[g]pteridine-2,4(1H,3H)-dione

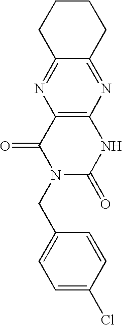

766 mg (2 mmol) 1-allyl-3-(4-chlorobenzyl)-6,7,8,9-tetrahydrobenzo[g]pteridine-2,4(1H,3H)-dione, ref. example 14, was dissolved in 15 mL dioxane in a dried 20 ml vial under nitrogen, then 160 μL (4.24 mmol) formic acid and 700 μL (5.03 mmol) triethylamine was added. The resulting solution was purged with nitrogen for 5 min, then 200 mg (0.173 mmol) Pd(PPh₃)₄ was added and purged for another 1 minute. The vial was sealed and heated to 100 C for 1 h using microwave heating. The solvents were evaporated and the residue was suspended in ethyl acetate/hexanes 1:3 and sonicated for 5 min, then filtered and the solid washed with the same solvent. The solid was dissolved in hot DCM/MeOH 9:1 and filtered hot through Celite. Solvents evaporated and residue dried. 664 mg (1.94 mmol, 96%) of the title compound was isolated as grey solid.

¹H-NMR (500 MHz, DMSO-d₆) δ 12.06 (br, 1H), 7.28-7.34 (m, 4H), 5.02 (s, 2H), 2.83-2.86 (m, 4H), 1.80-1.84 (m, 4H)

HRMS Calcd for [C₁₇H₁₅ClN₄O₂+H]⁺: 343.0962. Found: 343.0963.

### Step 2i

### Example 16

### 3-{[3-(4-chlorobenzyl)-2,4-dioxo-3,4,6,7,8,9-hexahydrobenzo[g]pteridin-1 (2H)-yl]methyl}benzonitrile was Synthesized According to Scheme 2

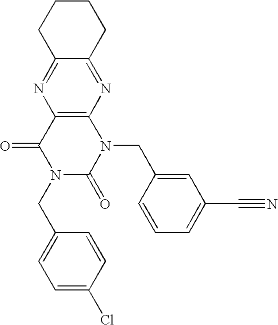

86 mg (0.25 mmol) 3-(4-chlorobenzyl)-6,7,8,9-tetrahydrobenzo[g]pteridine-2,4(1H,3H)-dione, Ref. Example 16 step 2h product, and 69 mg (0.5 mmol) K₂CO₃ were suspended in 2 mL DMF, then 66 mg (0.34 mmol) of 3-cyanobenzyl bromide was added. The resulting mixture was stirred at room temperature for 2 h 30 min. The reaction mixture was partitioned between 1N HCl and ethyl acetate. After phase separation the aqueous layer was extracted twice with ethyl acetate. The combined organic layers were dried over MgSO₄ and evaporated. The residue was recrystallized from DCM/hexanes. 98 mg (0.21 mmol, 85%) of the title compound was isolated.

¹H-NMR (400 MHz, CDCl₃) δ 7.75 (s, 1H), 7.70 (d, 7.8 Hz, 1H), 7.54 (d, 7.8 Hz, 1H), 7.48 (d, 8.5 Hz, 2H), 7.40 (t, 7.8 Hz, 1H), 7.25 (d, 8.5 Hz, 2H), 5.44 (s, 2H), 5.23 (s, 2H), 2.98-3.08 (m, 4H), 1.92-1.98 (m, 4H).

HRMS Calcd for [C₂₅H₂₀ClN₅O₂+H]⁺: 458.1384. Found: 458.1392.

Examples 17-20 were Prepared According to Example 16 Besides Described Exceptions:

### Example 17

### 3-(4-chlorobenzyl)-1-[2-(1H-pyrrol-1-yl)ethyl]-6,7,8,9-tetrahydrobenzo[g]pteridine-2,4(1H,3H)-dione

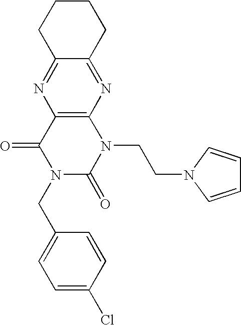

40 mg (0.117 mmol) 3-(4-chlorobenzyl)-6,7,8,9-tetrahydrobenzo[g]pteridine-2,4(1H,3H)-dione, ref. example 16 step 2h product, was dissolved in 1.5 mL DMF, then 48 mg (0.35 mmol) K₂CO₃ was added, followed by 41 mg (0.233 mmol) 1-(2-bromoethyl)pyrrole. The resulting mixture was stirred at room temperature overnight. The reaction mixture was diluted with 3 mL DCM and washed with 4 mL 1N HCl. The organic layer was filtered through a phase separator and evaporated. The residue was purified by reversed phase HPLC. 17 mg (0.039 mmol, 33%) of the title compound was isolated.

¹H-NMR (600 MHz, (CH₃)₂SO*, (CD₃)₂SO)) δ 7.33 (d, 8.5 Hz, 2H), 7.27 (d, 8.5 Hz, 2H), 6.57 (d, 1.8 Hz, 2H), 5.81 (d, 1.8 Hz, 2H), 5.06 (s, 2H), 4.44 (t, 6.2 Hz, 2H), 4.13 (t, 6.2 Hz, 2H), 2.85-2.91 (m, 4H), 1.80-1.84 (m, 4H).

HRMS Calcd for [C₂₃H₂₂ClN₅O₂+H]⁺: 436.1540. Found: 436.1536.

### Example 18

### 3-(4-chlorobenzyl)-1-(3,3-dimethyl-2-oxobutyl)-6,7,8,9-tetrahydrobenzo[g]pteridine-2,4(1H,3H)-dione

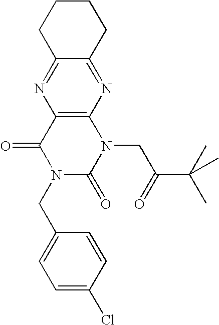

From 40 mg (0.117 mmol) 3-(4-chlorobenzyl)-6,7,8,9-tetrahydrobenzo[g]pteridine-2,4(1H,3H)-dione, ref. example 16 step 2h product, 27 mg (0.061 mmol, 52%) of the title compound was isolated.

¹H-NMR (600 MHz, (CH₃)₂SO*, (CD₃)₂SO)) δ 7.34 (d, 8.5 Hz, 2H), 7.30 (d, 8.5 Hz, 2H), 5.18 (s, 2H), 5.09 (s, 2H), 2.83-2.91 (m, 4H), 1.81-1.85 (m, 4H), 1.19 (s, 9H),

HRMS Calcd for [C₂₃H₂₅ClN₄O₃+H]⁺: 441.1693. Found: 441.1675.

### Example 19

### 3-(4-chlorobenzyl)-1-(3,3,3-trifluoro-2-hydroxypropyl)-6,7,8,9-tetrahydrobenzo[g]pteridine-2,4(1H,3H)-dione

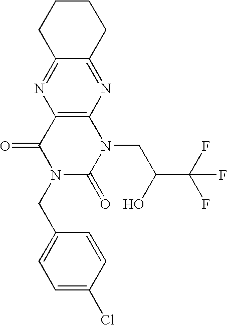

From 103 mg (0.3 mmol) 3-(4-chlorobenzyl)-6,7,8,9-tetrahydrobenzo[g]pteridine-2,4(1H,3H)-dione, ref. example 16 step 2h product, was dissolved in 2 mL DMF, then 83 mg (0.6 mmol) K₂CO₃ was added. The resulting mixture was stirred at room temperature for 5 min, then 3-bromo-1,1,1-trifluoro-2-propanol was added. The resulting mixture was stirred at room temperature for 24 h. The reaction mixture was diluted with 5 mL DCM and washed with 4 mL 1N HCl. The organic layer was filtered through a phase separator and evaporated. The residue was purified by reversed phase HPLC. Product containing fractions evaporated and residue suspended in water. Solid was filtered and washed with water. 24 mg (0.053 mmol, 17%) of the title compound was isolated as off-white solid.

¹H-NMR (500 MHz, CDCl₃) δ 7.47 (d, 8.5 Hz, 2H), 7.25 (d, 8.5 Hz, 2H), 5.26 (d, 13.7 Hz, 1H), 5.21 (d, 13.7 Hz, 1H), 4.58-4.69 (m, 2H), 4.34 (br, 1H), 3.70 (d, 7.5 Hz, 1H), 2.96-3.10 (m, 4H), 1.92-1.98 (m, 4H).

HRMS Calcd for [C₂₀H₁₈ClF₃N₄O₃+H]⁺: 455.1098. Found: 455.1070.

### Example 20

### 3-(4-chlorobenzyl)-1-propyl-6,7,8,9-tetrahydrobenzo[g]pteridine-2,4(1H,3H)-dione

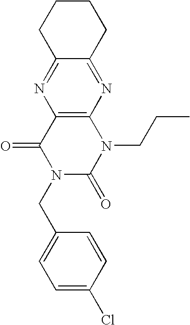

1.32 g (3.85 mmol) 3-(4-chlorobenzyl)-6,7,8,9-tetrahydrobenzo[g]pteridine-2,4(1H,3H)-dione, ref. example 16 step 2h product, was suspended in 10 mL DMF, then 1.08 g (7.78 mmol) K₂CO₃ was added. After 10 min stirring at room temperature, 600 μL (6.12 mmol) propyl iodide was added in one portion and stirring continued at room temperature. After 2 h the reaction mixture was partitioned between water and ethyl acetate. After phase separation, the organic layer washed with water, then dried over MgSO₄ and evaporated. The residue was purified by flash chromatography on silica using hexanes/ethyl acetate 50:50 as eluent. Product containing fractions were combined, evaporated and the residue crystallized from hexanes/ethyl acetate. 879 mg (2.28 mmol, 59%) of the title compound was isolated as colorless solid.

¹H-NMR (500 MHz, CDCl₃) δ 7.48 (d, 8.5 Hz, 2H), 7.23 (d, 8.5 Hz, 2H), 5.22 (s, 2H), 4.20 (t, 7.6 Hz, 2H), 2.94-3.08 (m, 4H), 1.91-1.97 (m, 4H), 1.64-1.74 (m, 2H), 0.93 (t, 7.6 Hz, 3H).

HRMS Calcd for [C₂₀H₂₁ClN₄O₂+H]⁺: 385.1431. Found: 385.1433.

### Step 2j

### Example 21

### 3-(4-chlorobenzyl)-1-propyl-6,7,8,9-tetrahydrobenzo[g]pteridine-2,4(1H,3H)-dione 5-oxide was Synthesized According to Scheme 2

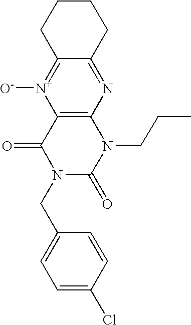

87 mg (0.23 mmol) 3-(4-chlorobenzyl)-1-propyl-6,7,8,9-tetrahydrobenzo[g]pteridine-2,4(1H,3H)-dione, ref. example 20, was dissolved in 5 mL dichloromethane, then 279 mg of 70% m-chloroperbenzoic acid was added. The resulting mixture was stirred at room temperature overnight, then washed with sat. NaHCO₃. The organic layer was filtered through a phase separator and evaporated. The residue was purified by preparative HPLC. 10 mg (0.025 mmol, 11%) of the title compound was isolated.

¹H-NMR (500 MHz, CDCl₃) δ 7.51 (d, 8.5 Hz, 2H), 7.27 (d, 8.5 Hz, 2H), 5.19 (s, 2H), 4.25 (t, 7.6 Hz, 2H), 2.84-2.93 (m, 4H), 1.85-1.93 (m, 4H), 1.69-1.78 (m, 2H), 0.98 (t, 7.6 Hz, 3H).

HRMS Calcd for [C₂₀H₂₁ClN₄O₃+H]⁺: 401.1380. Found: 401.1371.

### Step 2k

### Example 22

### 3-(4-chlorobenzyl)-1-propyl-7,8-dihydro-1H-cyclopenta[g]pteridine-2,4(3H,6H)-dione was Synthesized According to Scheme 2

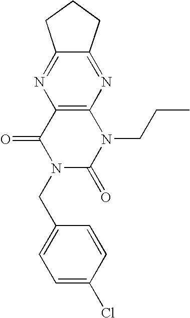

62 mg (0.2 mmol) 5,6-diamino-3-(4-chlorobenzyl)-1-propylpyrimidine-2,4(1H,3H)-dione, ref example 12 step 2e product, and 20 μL (0.2 mmol) 2-chlorocyclopentanone were dissolved in 1 mL methanol/acetic acid 9:1. The resulting mixture was heated to 160 C for 5 min using microwave heating. The solvents were evaporated, the residue dissolved in DCM and washed with sat. NaHCO₃. Organic layer filtered through a phase separator and evaporated. The residue was purified by reversed phase HPLC. 19 mg (0.051 mmol, 26%) of the title compound was isolated.

¹H-NMR (600 MHz, (CH₃)₂SO*, (CD₃)₂SO)) δ 7.32-7.34 (m, 4H), 5.09 (s, 2H), 4.09-4.13 (m, 2H), 3.05 (t, 7.6 Hz, 2H), 2.99 (t, 7.6 Hz, 2H), 2.13-2.19 (m, 2H), 1.58-1.65 (m, 2H), 0.85 (t, 7.5 Hz, 3H).

HRMS Calcd for [C₁₉H₁₉ClN₄O₂+H]⁺: 371.1275. Found: 371.1271.

### Step 2l

### Example 23

### 6-tert-butyl-3-(4-chlorobenzyl)-1-propylpteridine-2,4(1H,3H)-dione and 7-tert-butyl-3-(4-chlorobenzyl)-1-propylpteridine-2,4(1H,3H)-dione were Synthesized According to Scheme 2

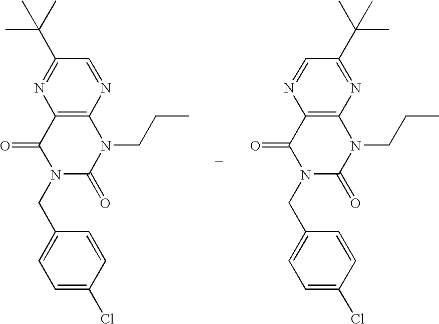

62 mg (0.2 mmol) 5,6-diamino-3-(4-chlorobenzyl)-1-propylpyrimidine-2,4(1H,3H)-dione, ref example 12 step 2e product, and 27 mg (0.2 mmol) 1-chlorpinacolin were dissolved in 1 mL methanol/acetic acid 9:1. The resulting mixture was heated to 160 C for 10 min using microwave heating. Then additional 10 mg (0.075 mmol) 1-chlorpinacolin was added and heating continued for 10 min. To the reaction mixture was added 2 drops of 30% H₂O₂ and stirred overnight. Then addition of 120 mg (3 mmol) NaOH to the reaction mixture. Then again addition of 2 drops of 30% H₂O₂. After 1 h reaction mixture diluted with DCM and washed with 1 N HCl. Organic phase filtered through a phase separator and evaporated. The residue was purified by reversed phase HPLC. 12 mg (0.031 mmol, 16%) of the title compound was isolated as a mixture of isomers.

¹H-NMR (600 MHz, (CH₃)₂SO*, (CD₃)₂SO)) δ 8.92 (s, 0.43H), 8.73 (s, 0.46H), 7.32-7.38 (m, 4H), 5.09-5.10 (m, 2H), 4.10-4.16 (m, 2H), 1.60-1.68 (m, 2H), 1.38 (s, 3.9H)1.36 (s, 5.1H), 0.85-0.90 (m, 3H).

HRMS Calcd for [C₂₀H₂₃ClN₄O₂+H]⁺: 387.1588. Found: 387.1580.

Biological Evaluation

Effects of the Compounds Acting as GABA_{B} Positive Allosteric Receptor Modulators (PAM) or Agonists in Functional in Vitro Assays

The effect of GABA in an automated GTPγS³⁵ radioligand filtration-binding assay in CHO cells expressing the GABA_{B(1A,2)} receptor heterodimer was studied in the presence or absence of the positive allosteric modulator test compounds. The positive allosteric modulator according to the invention increased both the potency and the efficacy of GABA.

The potency of the compounds i.e. the ability of the compounds to reduce the EC₅₀ of GABA was revealed by the concentration required to reduce GABA's EC₅₀ by 50%. The potency and efficacy of the compounds acting as agonists at the GABA_{B} receptor was also determined in a automated GTPγS³⁵ radioligand filtration-binding assay.

GTPγS35 Assay Principle

The GABA_{B} receptor is a G-protein coupled receptor. Binding of a ligand activates the receptor leading to recruitment of G-protein and a substitution of the G-protein bound GDP to GTP. The G-protein becomes active. The G-protein is inactivated by hydrolysis of GTP to GDP. G-proteins are membrane bound and therefore present in membrane preparations.

In the GTPγS³⁵ assay, GTP is not present but instead GTPγS³⁵ where one of the phosphate groups are substituted to a sulphur group which cannot be hydrolysed. Upon activation of the receptor, radiolabelled GTPγS³⁵ replaces the GDP. The complex cannot be inactivated and the radiolabelled complex is accumulating. At the end of the assay, the reaction mixture is filtered through a membrane-binding filter. Excess GTPγS³⁵ is removed by washing and the membrane bound S³⁵, which correlates to the degree of receptor activation, is measured with a β-Liquid Scintillation Counter.

Experimental Procedures

Materials and Reagents

HEPES, GDP, Trizma-HCl, Trizma Base, and Saponin were from Sigma-Aldrich; EDTA, NaCl and MgCl₂×6H₂O were from Merck; Sucrose was from BDH Laboratory supplies; EDTA was from USB Corporation; GABA was from Tocris; GTPγS³⁵ was from Amersham Radiochemicals (GE Healthcare); OptiPhase Supermix was from PerkinElmer; 384 well PS-microplates were from Greiner; 1.2 mL Square well storage plates, low profile were from Abgene; MultiScreen HTS 384 FB (1.0/0.65 μm) filter plates were from Millipore; Biomek AP96 P20 pipette tips (non sterile) were from Beckman; Nut mix F-12 (Ham), DMEM/F12, OptiMEM, penicillin/streptomycin solution (PEST), Lipofectamine, Zeocin, Hygromycin and Geneticin were from Invitrogen; FBS was from Hyclone. Accutase was from Innovative Cell Technologies.

Generation of Cell Lines Expressing the GABA_{B} Receptor

Cell Line Used for the Determination of the Test Compounds PAM Potency

GABA_{B}R1a and GABA_{B}R2 were cloned from human brain cDNA and subcloned into pCI-Neo (Promega) and pALTER-1 (Promega), respectively.

In order to optimise the Kozak consensus sequence of GABA_{B}R2, in situ mutagenesis was performed using the Altered Sites Mutagenesis kit according to manufacturer's instruction (Promega) with the following primer,

5′-GAATTCGCACCATGGCTTCCC-3′. The optimised GABA_{B}R2 was then restricted from pALTER-1 with Xho I+Kpn I and subcloned into the mammalian expression vector pcDNA3.1(−)/Zeo (Invitrogen) to produce the final construct, pcDNA3.1(−)/Zeo-GABA_{B}R2.

For generation of stable cell lines, CHO-K1 cells were grown in Nut mix F-12 (Ham) media supplemented with 10% FBS, 100 U/ml Penicillin and 100 μg/ml Streptomycin at 37° C. in a humidified CO₂-incubator. The cells were detached with 1 mM EDTA in PBS and 1 million cells were seeded in 100 mm petri dishes. After 24 hours the culture media was replaced with OptiMEM and incubated for 1 hour in a CO₂-incubator.

For generation of a cell line expressing the GABA_{B}R1a/GABA_{B}R2 heterodimer, GABA_{B}R1a plasmid DNA (4 μg) GABA_{B}R2 plasmid DNA (4 μg) and lipofectamine (24 μl) were mixed in 5 ml OptiMEM and incubated for 45 minutes at room temperature. The cells were exposed to the transfection medium for 5 hours, which then was replaced with culture medium. The cells were cultured for an additional 10 days before selection agents (300 μg/ml hygromycin and 400 μg/ml geneticin) were added. Twenty-four days after transfection, single cell sorting into 96-well plates by flow cytometry was performed using a FACS Vantage SE (Becton Dickinson, Palo Alto, Calif.). After expansion, the GABA_{B} receptor functional response was tested by measuring the GABA_{B} receptor dependent release of intracellular calcium in a fluorescence imaging plate reader (FLIPR). The clone with the highest functional response was collected, expanded and then subcloned by single cell sorting. The clonal cell line with the highest peak response in the FLIPR was used in the present study.

Cell Line Used for the Determination of the Test Compounds Agonist Potency

The human GABA_{B}R1a was subcloned into pIRESneo3 (Clontech) using GABA_{B}R1a construct as a template (refseqN NM001470). GABA_{B}R2 was subcloned into pCDNA5/FRT (Invitrogen) using GABA_{B}R2 construct as a template (refseqN NM005458). The Kozak sequence GCCACC was introduced before the start codon in both constructs.

For generation of stable cell lines, CHO K1 Flp-In cells (Invitrogen) were grown in DMEM/F 12 1:1 media supplemented with 10% FBS at 37° C. in a humidified CO₂-incubator. The cells were detached with Accutase and 1.5 million cells were seeded into T75 flasks. After 24 h, transfection of the cells were performed with the GABA_{B}R2 construct. For generation of cell lines expressing GABA_{B}R2, GABA_{B}R2 plasmid (1 μg) and pOG44 from Invitrogen (9 μg) were mixed with 30 μl Lipofectamine 2000 in 600 μl OptiMEM for 20 minutes. The cells were exposed to transfection medium for 5 hours and was then replaced with culture medium. After 2 days 0.5 mg/ml Hygromycin were added to culture medium. The cells were cultured for an additional 10 days to establish a stable cell mix expressing GABA_{B}R2. For generation of a cell line expressing the GABA_{B}R1a/GABA_{B}R2 heterodimer, GABA_{B}R1a plasmid DNA (8 μg) and Lipofectamine (30 μl) were mixed in 600 μl OptiMEM and incubated for 20 minutes before added to CHO-Flp-In cells expressing GABA_{B}R2. After 2 days additional selection agent was added (0.8 mg/ml Geneticin). The cells were cultured for another 10 days to generate a stable mixed population expressing the GABA_{B}R1a/GABA_{B}R2 heterodimer. The cell line was analyzed by GTPγS³⁵ assay with GABA as agonist.

GTPγS³⁵ Assay for Determination of PAM Potency

GTPγS³⁵ radioligand filtration-binding assays were performed using an automated workstation at 30° C. for 1 hour in assay buffer (50 mM HEPES, 40 mM NaCl, 1 mM MgCl₂×6H₂O, 30 μg/mL Saponin, pH 7.4 at RT) containing 0.025 μg/μL of membrane protein (prepared from the cell line described above), 10 μM GDP and 0.55 nCi/μL GTPγS³⁵ in a final volume of 60 μL. The reaction was started by the addition of serially diluted GABA (final start concentration 1 mM dilution factor 3) in the presence or absence of four concentrations (final conc 10, 1, 0.1 and 0.01 μM) of PAM. The reaction was terminated and membranes collected by addition of ice-cold wash buffer (50 mM Tris-HCl, 5 mM MgCl₂×6H₂O, 50 mM NaCl, pH 7.4 at 4° C.) followed by rapid filtration under vacuum through a MultiScreen HTS 384 FB filter plate. Repeated washing of the filters with ice-cold wash buffer washed the unbound radioligand away. The filter plates were dried for 1½-2 hours at 50° C., then 8 μL scintillation liquid was added per well followed by incubation at RT for at least 20 minutes before bound radioactivity was determined using a β-Liquid Scintillation Counter (1450 Microbeta Trilux, Wallac, Finland)

GTPγS³⁵ Assay for Determination of Agonist Potency

GTPγS³⁵ radioligand filtration-binding assays were performed using an automated workstation at 30° C. for 1 hour in assay buffer (50 mM HEPES, 40 mM NaCl, 1 mM MgCl₂×6H₂O, 30 μg/mL Saponin, pH 7.4 at RT) containing 0.025 μg/μL of membrane protein (prepared from the cell line described above), 10 μM GDP and 0.55 nCi/μL GTPγS³⁵ in a final volume of 60 μL. The reaction was started by the addition of compounds (GABA was always included as a positive control), start concentration 100 μM dilution factor 3. The reaction was terminated and membranes collected by addition of ice-cold wash buffer (50 mM Tris-HCl, 5 mM MgCl₂×6H₂O, 50 mM NaCl, pH 7.4 at 4° C.) followed by rapid filtration under vacuum through a MultiScreen HTS 384 FB filter plate. Repeated washing of the filters with ice-cold wash buffer washed the unbound radioligand away. The filter plates were dried for 1½-2 hours at 50° C., then 8 μL scintillation liquid was added per well followed by incubation at RT for at least 20 minutes before bound radioactivity was determined using a β-Liquid Scintillation Counter (1450 Microbeta Trilux, Wallac, Finland)

Calculation and Interpretation of Results

Controls

100% activation (max) is calculated as the mean value for wells containing 1 mM GABA. 0% activation (min) is calculated as the mean value for the wells with DMSO added instead of compound.

Calculation of Results

All values are calculated as Compound % activation=100*[(X−min)/(max−min)], where X is representing raw value for the compound.

Test Compound PAM Potency.

EC₅₀, max, min and slope values were calculated from GABA dose-response curves in the presence and absence of PAM constructed using a 4 Parameter Logistic Model (A+((B−A)/(1+((C/x)^{D})))) with XLfit (Model 205, Version 4.2.2, IDBS Solutions), where C=EC₅₀ and D=Slope Factor.

The potency (PAM EC₅₀) of the PAM in GTPγS assays was determined by plotting the log EC₅₀ for GABA against the four log concentrations of the positive allosteric modulator in the presence of which the measurement was performed, using the 4 Parameter Logistic Model described above (slope fixed to 1).

Test Compound Agonist Potency.

EC₅₀, max, min and slope values were calculated from compound (or GABA) concentration response curves constructed using a 4 Parameter Logistic Model (A+((B−A)/(1+((C/x)^{D})))) with XLfit (Model 205, Version 4.2.2, IDBS Solutions), where C=EC₅₀ and D=Slope Factor.

Generally, the potency of the compounds of formula (I) ranges from EC₅₀s between 40 μM and 0.001 μM. Hereinbelow, individual EC₅₀ values are presented.

| | Mean agonist | Mean (EC₅₀ derived) |
|---|---|---|
| Compound | EC₅₀ (μM) | PAM EC₅₀ (μM) |
| Example 1 | — | 6.0 μM |
| Example 2 | — | 4.3 μM |
| Example 3 | — | 0.6 μM |
| Example 4 | — | 0.6 μM |
| Example 5 | — | 2.9 μM |
| Example 6 | 4.7 μM | 0.8 μM |
| Example 7 | — | 5.1 μM |
| Example 8 | — | 2.4 μM |
| Example 9 | — | 7.0 μM |
| Example 10 | — | 0.8 μM |
| Example 11 | — | 1.3 μM |
| Example 12 | 1.4 μM | — |
| Example 13 | 0.8 μM | — |
| Example 14 | 1.6 μM | — |
| Example 15 | 36.8 μM | 1.3 μM |
| Example 16 | 1.0 μM | — |
| Example 17 | 1.6 μM | — |
| Example 18 | 0.5 μM | — |
| Example 19 | 1.3 μM | — |
| Example 20 | 0.8 μM | 0.8 μM |
| Example 21 | 7.7 μM | — |
| Example 22 | 3.7 μM | — |
| Example 23 | 2.2 μM | — |

1. A compound of the formula 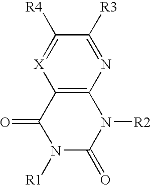
or a pharmaceutically acceptable salt thereof, wherein:
R¹ is aryl-C₁-C₁₀-alkyl, wherein the aryl is substituted by halogen;
R² is selected from the group consisting of C₁-C₁₀-alkyl unsubstituted or optionally substituted by one or more of oxo, hydroxy, halogen and tri-C₁-C₁₀-alkylsilyl; C₂-C₁₀-alkenyl; C₃-C₁₀-cycloalkyl-C₁-C₁₀-alkyl; aryl-C₁-C₁₀-alkyl unsubstituted or optionally substituted by one or more of C₁-C₁₀-alkoxy, halogen, cyano and aroyl; and heteroaryl-C₁-C₁₀-alkyl;
R³ is selected from the group consisting of hydrogen and C₁-C₁₀-alkyl;
R⁴ is selected from the group consisting of hydrogen and C₁-C₁₀-alkyl;
or R³ and R⁴ together with the carbon atoms bonded thereto form a non-aromatic 5-membered ring unsubstituted or optionally substituted by one or more of C₁-C₁₀-alkyl; or together form a non-aromatic 6-membered ring;
and X is selected from the group consisting of N and N-oxide.

2. The compound according to claim 1, wherein:
R¹ is from benzyl substituted by chloro;
R² is selected from the group consisting of propyl; allyl; 3,3-dimethylbutyl; 2-oxo-3,3-dimethylbutyl; 2-hydroxy-3,3,3-trifluoropropyl; 2-trimethylsilylethyl; cyclohexylmethyl; benzyl unsubstituted or optionally substituted by one or more of chloro, methoxy, cyano, and benzoyl; 2-phenylethyl; and 2-(1H-pyrrol-1-yl)ethyl;
R³ is selected from the group consisting of hydrogen, methyl, ethyl, and tert-butyl;
R⁴ is selected from the group consisting of hydrogen, methyl, ethyl, and tert-butyl;
or R³ and R⁴ together with the carbon atoms bonded thereto form a non-aromatic 5-membered ring, optionally unsubstituted or substituted by one or more of methyl; or together form a non-aromatic 6-membered ring.

3. The compound according to claim 1, which is selected from the group consisting of:
3-(4-chlorobenzyl)-1-(2,4-dimethoxybenzyl)pteridine-2,4(1H,3H)-dione;
3-(4-chlorobenzyl)-1-propylpteridine-2,4(1H,3H)-dione;
3-(4-chlorobenzyl)-1-[2-(trimethylsilyl)ethyl]pteridine-2,4(1H,3H)-dione;
3-(4-chlorobenzyl)-1-(cyclohexylmethyl)pteridine-2,4(1H,3H)-dione;
3-(4-chlorobenzyl)-1-(3,3-dimethyl-2-oxobutyl)pteridine-2,4(1H,3H)-dione;
3-(4-chlorobenzyl)-1-(3,3-dimethylbutyl)pteridine-2,4(1H,3H)-dione;
1-benzyl-3-(4-chlorobenzyl)pteridine-2,4(1H,3H)-dione;
1,3-bis(4-chlorobenzyl)pteridine-2,4(1H,3H)-dione;
3-(4-chlorobenzyl)-1-(4-methoxybenzyl)pteridine-2,4(1H,3H)-dione;
1-(4-benzoylbenzyl)-3-(4-chlorobenzyl)pteridine-2,4(1H,3H)-dione;
3-(4-chlorobenzyl)-1-(2-phenylethyl)pteridine-2,4(1H,3H)-dione;
3-(4-chlorobenzyl)-6,8-dimethyl-1-propyl-7,8-dihydro-1H-cyclopenta[g]pteridine-2,4(3H,6H)-dione;
3-(4-chlorobenzyl)-6,7-diethyl-1-propylpteridine-2,4(1H,3H)-dione;
1-allyl-3-(4-chlorobenzyl)-6,7,8,9-tetrahydrobenzo[g]pteridine-2,4(1H,3H)-dione;
3-(4-chlorobenzyl)-6,7-dimethyl-1-propylpteridine-2,4(1H,3H)-dione;
3-{[3-(4-chlorobenzyl)-2,4-dioxo-3,4,6,7,8,9-hexahydrobenzo[g]pteridin-1(2H)-yl]methyl}benzonitrile;
3-(4-chlorobenzyl)-1-[2-(1H-pyrrol-1-yl)ethyl]-6,7,8,9-tetrahydrobenzo[g]pteridine-2,4(1H,3H)-dione;
3-(4-chlorobenzyl)-1-(3,3-dimethyl-2-oxobutyl)-6,7,8,9-tetrahydrobenzo[g]pteridine-2,4(1H,3H)-dione;
3-(4-chlorobenzyl)-1-(3,3,3-trifluoro-2-hydroxypropyl)-6,7,8,9-tetrahydrobenzo[g]pteridine-2,4(1H,3H)-dione;
3-(4-chlorobenzyl)-1-propyl-6,7,8,9-tetrahydrobenzo[g]pteridine-2,4(1H,3H)-dione;
3-(4-chlorobenzyl)-1-propyl-6,7,8,9-tetrahydrobenzo[g]pteridine-2,4(1H,3H)-dione 5-oxide;
3-(4-chlorobenzyl)-1-propyl-7,8-dihydro-1H-cyclopenta[g]pteridine-2,4(3H,6H)-dione;
6-tert-butyl-3-(4-chlorobenzyl)-1-propylpteridine-2,4(1H,3H)-dione; and
7-tert-butyl-3-(4-chlorobenzyl)-1-propylpteridine-2,4(1H,3H)-dione.

4. (canceled)

5. (canceled)

6. A pharmaceutical composition comprising a compound according to any one of claims 1 to 3 as an active ingredient and a pharmaceutically acceptable carrier or diluent.

7. A method for the treatment or inhibition of gastroesophageal reflux disease (GERD), the method comprising administering a therapeutically effective amount of a compound according to any one of claims 1 to 3, optionally in combination with a GABA_{B} receptor agonist, to a patient in need thereof.

8. A method for the treatment or inhibition of reflux, the method comprising administering a therapeutically effective amount of a compound according to any of claims 1 to 3, optionally in combination with a GABA_{B} receptor agonist, to a patient in need thereof.

9. A method for the treatment or inhibition of transient lower esophageal sphincter relaxations (TLESRs), the method comprising administering a therapeutically effective amount of a compound according to any one of claims 1 to 3, optionally in combination with a GABA_{B} receptor agonist, to a patient in need thereof.

10. A method for the treatment or inhibition of a functional gastrointestinal disorder, the method comprising administering a therapeutically effective amount of a compound according to any one of claims 1 to 3, optionally in combination with a GABA_{B} receptor agonist, to a patient in need thereof.

11. The method according to claim 10, wherein said functional gastrointestinal disorder is functional dyspepsia.

12. A method for the treatment or inhibition of irritable bowel syndrome (IBS), the method comprising administering a therapeutically effective amount of a compound according to claims 1 to 3, optionally in combination with a GABA_{B} receptor agonist, to a patient in need thereof.

13. The method according to claim 12, wherein said IBS is constipation predominant IBS.

14. The method according to claim 12, wherein said IBS is diarrhea predominant IBS.

15. The method according to claim 12, wherein said IBS is alternating bowel movement predominant IBS.
